(19) United States
(12) Patent Application Publication
(10) Pub. No.: US 2009/0269753 A1
(43) Pub. Date: Oct. 29, 2009

(54) GENOTYPING FOR SRC-1 PREDICTS FOR BONE LOSS

(75) Inventors: Steffi Oesterreich, Missouri City, TX (US); Alexandra Richter, Houston, TX (US); Adrian Lee, Missouri City, TX (US); C. Kent Osborne, Houston, TX (US); Sean E. Mcguire, Houston, TX (US); Ryan Hartmaier, Houston, TX (US)

Correspondence Address: FULBRIGHT & JAWORSKI, LLP, 1301 MCKINNEY, SUITE 5100, HOUSTON, TX, 77010-3095, US

(73) Assignee: BAYLOR COLLEGE OF MEDICINE, Houston, TX (US)

(21) Appl. No.: 12/275,195

(22) Filed: Nov. 20, 2008

Related U.S. Application Data
(60) Provisional application No. 61/073,421, filed on Jun. 18, 2008.
(60) Provisional application No. 60/991,138, filed on Nov. 29, 2007.

(51) Int. Cl. C12Q 1/68; C12Q 1/48

(52) U.S. Cl. 435/6; 435/15

(57) ABSTRACT

Osteoporosis is a common skeletal disease characterized by loss of bone mineral density (BMD) and increased risk of fracture. Osteoporosis most commonly occurs in postmenopausal women due to estrogen deficiency. We identified 3 genetic variants in steroid receptor coactivator 1 (SRC-1) that are significantly associated with a decrease in BMD in women. We characterized a functional variant in exon 18 of SRC-1 that is associated with increased loss of bone mineral density in women who received tamoxifen for treatment or prevention of breast cancer. In vitro experiments show that this variant decreases estrogen receptor alpha response (ER-alpha) to hormone, suggesting an attenuated response to endogenous and exogenous hormones in the bone of these women, and therefore a need for additional bone protective measures.

### CROSS-REFERENCE TO RELATED APPLICATIONS

This application claims under 35 U.S.C. § 119(e) the benefit of U.S. provisional patent application 61/073,421, filed 18 Jun. 2008. This application also claims under 35 U.S.C. § 119 (e) the benefit of U.S. provisional patent application 60/991,138, filed 29 Nov. 2007. The contents of the foregoing two provisional patent applications are hereby incorporated by reference in their entireties.

### STATEMENT REGARDING FEDERALLY SPONSORED RESEARCH OR DEVELOPMENT

This work was supported in part by a Pharmacogenetics Research Network Grant # U-01 GM61373 (DAF) which supports the Consortium on Breast Cancer Pharmacogenomics (COBRA). Other support came from Pilot Project (SO) from SPORE (CA58183) (CKO), Clinical Pharmacology training Grant 5T32-GM08425 from the National Institute of General Medical Sciences, National Institutes of Health (Bethesda, Md.), CA112403 (JX), GCRC # M01-RR000042 (University of Michigan), M01-RR13297, M01-RR020359 (Georgetown University), and M01-RR00750 (Indiana University) from the National Center for Research Resources (NCRR), a component of the National Institutes of Health (NIH). The United States Government may have certain rights to patent(s) issuing on the invention(s) disclosed herein pursuant to 35 U.S.C. § 200, et seq.

### BACKGROUND OF THE INVENTION

Osteoporosis, characterized by progressive loss of Bone Mass Density (BMD), is one of the most common disorders in the elderly. As a strong predictor for fracture risk, osteoporosis is associated with high morbidity and mortality¹. Up to 40% of postmenopausal women are affected², primarily because of estrogen deficiency after menopause³. Maintaining bone mass is determined by a balance between bone formation by osteoblasts and bone resorption by osteoclasts. Decreased estrogen levels after menopause lead to an increase in osteoclast numbers and increased bone resorption. Estrogens and selective estrogen receptor modulators (SERMs), such as tamoxifen and raloxifene, suppress osteoclast formation and inhibit bone loss³.

Recent studies in knockout (−/−) mice lacking Estrogen Receptor-alpha (ER-alpha) expression specifically in osteoblasts or osteoclasts have demonstrated a critical role for ER-alpha in regulating BMD^{4,5}. Nakamura et al.⁵ showed that osteoclast-specific ER-alpha female −/− mice developed trabecular bone loss, which is also observed in osteoporotic postmenopausal women. Further, they demonstrated that estrogen, as well as tamoxifen, shows an osteoprotective effect by increasing the apoptosis rate of osteoclasts. Krum et al.⁴ examined the mechanism by which estrogen induces osteoclast apoptosis. They described a paracrine mechanism through upregulation of FasL in osteoblasts by estrogen and subsequent apoptosis of pre-osteoclasts, resulting in reduced bone resorption and a relative increase in osteoblast activity⁴. Estrogen withdrawal, as occurs in menopause, leads to a down-regulation of FasL and an increase in osteoclast numbers, causing increased bone resorptive activity.

Genetic risk factors play a major role in the pathogenesis of osteoporosis⁶. Recent genome-wide association studies (GWAS)⁷⁻⁹ identified a number of loci associated with decreased BMD and osteoporotic fractures including single nucleotide polymorphisms (SNPs) in osteoprotegerin, lipoprotein-receptor-related protein LRP5, RANKL, and ERα. Styrkarsdottir et al.⁸ identified 5 variants in ER-alpha (rs9479055, rs4870044, rs1038304, rs6929137, rs19999805) with minimal linkage disequilibrium (LD), which were associated with hip BMD. The Framingham Osteoporosis Study, which examined the association of ten quantitative bone traits with genotyping data acquired from a genome-wide scan performed in participants of the Framingham Heart Study (FHS), identified 3 intronic SNPs in ER-alpha (rs1884052, rs3778099, and rs3866461) that were associated with bone mass and geometry⁹. And finally, the Genetic Markers for Osteoporosis (GENOMOS) study examined 3 common SNPs in intron 1 of ERα in a cohort of 18,917 individuals and found no significant association with BMD. Interestingly, they did find a significant reduction in fracture risk for one of the SNPs (rs9340799), although the mechanism for this reduction in risk fracture remains unclear¹⁰. These data clearly suggest a complex pattern of association between polymorphisms at the ER-alpha locus and BMD, and that more studies are necessary to understand the mechanism(s) whereby these SNPs exert their effects.

Members of the p160 class of steroid receptor coactivators are major regulators of steroid hormone receptor activity¹¹. The first member of this class to be identified was SRC-1¹², which has been shown to interact with and coactivate several nuclear receptors, including ER-alpha¹³. SRC-1 has been shown to play an important role in mediating the relative agonist/antagonist activities of the SERM tamoxifen¹⁴ resulting in antagonist properties of tamoxifen in the breast and agonist properties in the endometrium¹⁵ and in bone¹⁶. Importantly, studies in mice lacking SRC-1 have revealed increased bone turnover and osteopenia, similar to the effects of estrogen deficiency, that were refractory to the administration of exogenous estrogen^{17,18}. These studies demonstrated a critical role for SRC-1 in bone metabolism, and therefore identified it as a candidate gene for human osteoporosis.

### BRIEF SUMMARY OF THE INVENTION

In one embodiment, the present disclosure provides a method of detecting a nucleic acid polymorphism in SRC-1 comprising the step of detecting an allele of: a) nucleic acid polymorphism in linkage disequilibrium (LD) with the SRC-1 nucleic acid polymorphism, or b) the nucleic acid polymorphism in SRC-1.

In one embodiment, the present disclosure provides a method according to paragraph comprising the step of analyzing a nucleic acid polymorphism in linkage disequilibrium (LD) with the SRC-1 nucleic acid polymorphism, wherein the polymorphism in linkage disequilibrium (LD) with the SRC-1 nucleic acid polymorphism belongs to a haplotype containing the nucleic acid polymorphism in SRC-1

In one embodiment, the present disclosure provides a method according to paragraph [0006] or [0007], wherein the polymorphism linkage disequilibrium (LD) with the SRC-1 nucleic acid polymorphism is a SNP.

In one embodiment, the present disclosure provides a method according to paragraph [0006] comprising the step of analyzing the nucleic acid polymorphism in SRC-1, wherein the nucleic acid polymorphism in SRC-1 is a SNP.

In one embodiment, the present disclosure provides a method according to paragraph [0006]-[0008], or [0009], wherein the allele of the nucleic acid polymorphism in SRC-1 encodes a Serine at amino acid 1272 of the protein encoded by SRC-1, or at an equivalent amino acid position in a homologue of SRC-1.

In one embodiment, the present disclosure provides a method according to paragraph [0010], wherein the nucleic acid polymorphism in SRC-1 is an SNP rs1804645.

In one embodiment, the present disclosure provides a method according to paragraph [0006]-[0011], or [0009], wherein the nucleic acid polymorphism in SRC-1 is an SNP rs2083389 and/or an SNP rs719189.

In one embodiment, the present disclosure provides a method according to paragraph [0006]-[0011], or [0012] wherein the nucleic acid comprising SRC-1 is derived from a blood sample or other bodily tissue sample.

In one embodiment, the present disclosure provides a method according to paragraph [0006]-[0012] or [0013] comprising the step of amplifying a genomic DNA comprising the SRC-1 nucleic acid polymorphism, the amplified genomic DNA optionally further including the nucleic acid polymorphism in linkage disequilibrium (LD) with the SRC-1 nucleic acid polymorphism.

In one embodiment, the present disclosure provides a method according to paragraph [0014] wherein the step of amplifying the genomic DNA comprises a polymerase chain reaction.

In one embodiment, the present disclosure provides a method of determining the risk of osteopenia or osteoporosis in a subject comprising the steps of: a) detecting in the subject, a nucleic acid polymorphism in SRC-1 according to the method of any of paragraph [0006]-[0014] or [0015], and b) correlating the detected allele with a risk of the subject developing osteopenia or osteoporosis.

In specific embodiments of the method of [0016], the subject has one or more of the following characteristics: the subject is human, the subject is female, the subject has been or will be exposed to an antiestrogen, the subject has not been exposed to an antiestrogen, the subject has not been provided hormone replacement therapy, and/or the subject has breast or prostate cancer.

In specific embodiments of the method of [0017] involving antiestrogen, the antiestrogen is one or more of tamoxifen, raloxifene, goserelin acetate, leuprolide acetate, megestrol, toremifene, fulvestrant; a nonsteroidal or a steroidal aromatase inhibitor including, for example, exemestane, anastrozole and letrozole.

In one embodiment, the present disclosure provides a method of screening an SRC-1 protein variant for modulation of a coactivation potential of the variant comprising the step of measuring a transcription of a nucleic acid, or a protein expression from the transcription of the nucleic acid, in the presence of the SRC-1 protein variant.

In one embodiment, the present disclosure provides a method according to paragraph [0019], wherein the nucleic acid and SRC-1 protein variant are in a cell, cell lysate, or cell lysate fraction.

In one embodiment, the present disclosure provides a method according to paragraph [0020] wherein the nucleic acid and SRC-1 protein variant are in an estrogen responsive cell.

In one embodiment, the present disclosure provides a method according to paragraph [0021], further comprising the step of exposing the estrogen responsive cell to one or more of estradiol, tamoxifen, raloxifene, goserelin acetate, leuprolide acetate, megestrol, toremifene, fulvestrant; a nonsteroidal or a steroidal aromatase inhibitor including, for example, exemestane, anastrozole and letrozole.

In one embodiment, the present disclosure provides a method according to paragraph [0019]-[0021], or [0022], wherein the nucleic acid comprises a reporter gene and the nucleic acid is capable of being transactivated by an estrogen receptor.

In one embodiment, the present disclosure provides a method according to paragraph [0019]-[0022], or [0023] wherein the cell is an MCF-7 cell, an SRC-1−/− osteoclast cell or an SRC-1−/− osteoclast-like cell.

In one embodiment, the present disclosure provides a method according to paragraph [0023] or [0024], wherein the reporter gene encodes a luciferase.

In one embodiment, the present disclosure provides a method according to paragraph [0019]-[0024], or [0025] further comprising the steps of: a) measuring a transcription of a nucleic acid, or a protein expression from the transcription of the nucleic acid, in the presence of a SRC-1 protein having a Proline at amino acid 1272, and b) comparing the measurements for the SRC-1 protein having a Proline at amino acid 1272 and the measurements for the SRC-1 protein variant to determine the modulation of the coactivation potential of the SRC-1 protein variant.

In one embodiment, the present disclosure provides a method according to paragraph [0019]-[00245], or [0026], further comprising the steps of: a) screening the SRC-1 protein variant for modulation of the coactivation potential in the presence of an additional compound, and b) determining whether the additional compound modulates the coactivation potential of the SRC-1 protein variant.

In one embodiment, the present disclosure provides a method of screening an SRC-1 protein variant for a modulation of a coactivation potential of the variant comprising the step of measuring an expression of a Fas ligand on a cell, the cell comprising an SRC-1 protein variant.

In one embodiment, the present disclosure provides a method according to paragraph [0028], further comprising the steps of: a) measuring an expression of a Fas ligand on a second cell, the second cell comprising an SRC-1 protein having a Proline at amino acid 1272, and b) comparing the measurements for the SRC-1 protein having a Proline at amino acid 1272 and the SRC-1 protein variant to determine the modulation of the coactivation potential of the SRC-1 protein variant.

In one embodiment, the present disclosure provides a method according to paragraph [0028] or [0029], further comprising the step of exposing the cell to one or more of estradiol, tamoxifen, raloxifene, goserelin acetate, leuprolide acetate, megestrol, toremifene, fulvestrant; a nonsteroidal or a steroidal aromatase inhibitor including, for example, exemestane, anastrozole and letrozole.

In one embodiment, the present disclosure provides a method according to paragraph [0028], [0029] or [0030] wherein the cell is an SRC-1−/− osteoclast cell or an SRC-1−/− osteoclast-like cell.

In one embodiment, the present disclosure provides a method according to paragraph [0028]-[0030] or [0031], further comprising the steps of: a) screening the SRC-1 protein variant for modulation of the coactivation potential in the presence of an additional compound, and b) determining whether the additional compound modulates the coactivation potential of the SRC-1 protein variant.

One specific embodiment of the present disclosure is the use of one or more nucleic acid polymorphisms within the human SRC-1 gene or in linkage disequilibrium with the human SRC-1 gene for general female population screening to assess the risk of osteopenia and/or osteoporosis. In particular embodiments, the test may be performed using one or more of the specific SRC-1 SNPs identified herein, for example on a DNA chip.

### BRIEF DESCRIPTION OF THE SEVERAL VIEWS OF THE DRAWINGS

FIG. 1: Schematic presentation of functional domains of SRC-1, and localization of the SRC-1 SNP P1272S.

FIG. 2: ER-alpha coactivation is attenuated in the presence of SRC-1 SNP (P1272S). MCF-7 cells were transiently transfected with ERE-TK-Luc reporter constructs and expression constructs for empty vector (control) or WT or SNP (P1272S) SRC-1. Cells were treated with vehicle or estradiol (10 nM), and relative luciferase units (RLU) were determined by correcting for co-transfected beta-gal. The data is presented as fold over control relative to untreated vehicle. Error bars represent standard deviation. *p<0.05 (two-way ANOVA test).

FIG. 3: SRC-1 SNP (P1272S) results in decreased ER activity in SRC-1 −/− osteoclasts as compared to SRC-1 WT. Transfection of SRC-1 WT resulted in a significant increase of ER activity after treatment with either Estrogen or Tamoxifen, suggesting that Tamoxifen acts as an ER agonist in these cells. In contrast, this Estrogen and Tamoxifen response was impaired in the presence of SRC-1 SNP (P1272S).

FIG. 4: Estrogen fails to induce apoptosis in the presence of SRC-1 P1272S. Cells were transfected with empty vector (control), SRC-1 WT, or SRC-1 P1272S expression constructs and then treated with either vehicle or estrogen (10 nM) for 16 hours. Cells were then fixed and apoptosis was detected by determining the ratio of TUNEL-positive cells and DAPI-counterstained cells.

FIG. 5: Decreased BMD in P1272S carriers receiving tamoxifen. Patients carrying the P1272S SNP allele showed a decrease in lumbar BMD (n=4) of 6.4% (p=0.04) and hip BMD (n=4) of 4.3% (p=0.08), Error bars represent SEM.

FIG. 6: Decreased BMD associated with alleles of rs2083389.

### DETAILED DESCRIPTION OF THE INVENTION

To investigate the role of variations at the human SRC-1 locus in maintenance of BMD, we initially sought to identify SNPs in the coding regions of SRC-1. First, we carried out direct sequencing of all SRC-1 coding exons in 48 Caucasian and 48 African-American apparently normal individuals to identify novel SNPs, and to gain population-specific SNP information (Coriell Institute, NJ). From this effort we identified a total of six variants (Table 1). SNPs were identified through dbSNP (http://www.ncbi.nlm.nih.gov/projects/SNP/) and by full exon sequencing of (Coriell samples. Our re-sequencing is listed as “Coriell re-sequencing” with AA representing African Americans and CA representing Caucasian Americans. (AGI ASP population is a mixture of African American and Caucasian samples. CEPH, HapMap-CEU, and AFD_EUR_PANEL represent populations of European descent. HapMap-HCB and AFD_CHN_PANEL populations are of Chinese descent. HapMap-YRI and AFD_AFR_PANEL are populations of African descent. HapMap-JPT is a population of Japanese descent. MAF=minor allele frequency).

TABLE 1
SNPs in SRC-1 coding regions.

| | Amino | Amino | | | | |
|---|---|---|---|---|---|---|
| | Acid | Acid | | | Samples | |
| rs# | Position | Change | Alleles | Population | (indiv) | MAF |
| rs1804645 | 1272 | P/S | C/T | CEPH | 92 | N/A |
| | | | | AGI_ASP population | 37 | 0.014 |
| | | | | Coriell resequencing - AA | 48 | 0.010 |
| | | | | Coriell resequencing - CA | 48 | 0.031 |
| rs11125763 | 1267 | L/L | T/G | AGI_ASP population | 37 | 0.189 |
| | | | | HapMap-CEU | 59 | 0.110 |
| | | | | HapMap-HCB | 45 | 0.000 |
| | | | | HapMap-JPT | 45 | 0.011 |
| | | | | HapMap-YRI | 60 | 0.500 |
| | | | | Coriell resequencing - AA | 48 | 0.292 |
| | | | | Coriell resequencing - CA | 48 | 0.073 |
| rs13430401 | 1068 | L/L | T/C | HapMap-CEU | 60 | 0.000 |
| | | | | HapMap-HCB | 45 | 0.000 |
| | | | | HapMap-JPT | 45 | 0.000 |
| | | | | HapMap-YRI | 60 | 0.000 |
| | | | | Coriell resequencing - AA | 48 | 0.031 |
| | | | | Coriell resequencing - CA | 48 | 0.000 |
| | | | | No Frequency Data in dbSNP | | |
| rs41281515 | 641 | A/A | C/T | Coriell resequencing - AA | 47 | 0.000 |
| | | | | Coriell resequencing - CA | 48 | 0.010 |
| | | | | Not found in dbSNP | | |
| No rs# | 177 | L/L | A/G | Coriell resequencing - AA | 48 | 0.010 |
| | | | | Coriell resequencing - CA | 47 | 0.000 |
| rs11125744 | 154 | T/T | G/C | AFD_EUR_PANEL | 24 | 0.104 |
| | | | | AFD_AFR_PANEL | 23 | 0.391 |
| | | | | AFD_CHN_PANEL | 24 | 0.000 |
| | | | | AGI_ASP population | 38 | 0.276 |
| | | | | HapMap-CEU | 60 | 0.100 |
| | | | | HapMap-HCB | 45 | 0.000 |
| | | | | HapMap-JPT | 44 | 0.012 |
| | | | | HapMap-YRI | 60 | 0.567 |
| | | | | Coriell resequencing - AA | 48 | 0.417 |
| | | | | Coriell resequencing - CA | 47 | 0.128 |

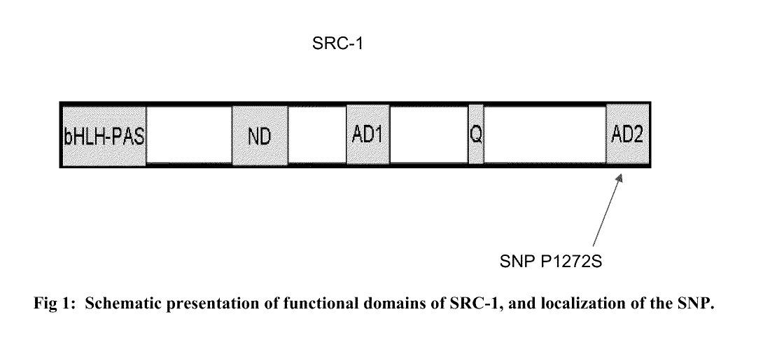

Of all the variants identified, rs1804645 represented the only non-synonymous SNP, changing proline to serine at amino acid position 1272 in exon 18; it will therefore be referred to as P1272S (FIG. 1).

We have found that the genetic variant P1272S of human SRC-1 decreases its activity and is associated with increased bone loss as detected by decreased bone mineral density (BMD). The variation is located in the c-terminus of SRC-1, at nucleotide 3814, resulting in an amino acid change from Proline to Serine at amino acid 1272 (FIG. 1). Information on this SNP (rs1804645) can be found on publicly available data bases, such as the dbSNP database. To verify this sequence information, and to obtain a better idea on frequency of this variant, we genotyped SRC-1 in 96 control DNA samples obtained from the Corriell Institute. We found this SNP in 3 Caucasian and 1 African American women, thus the frequency is approximately 5%.

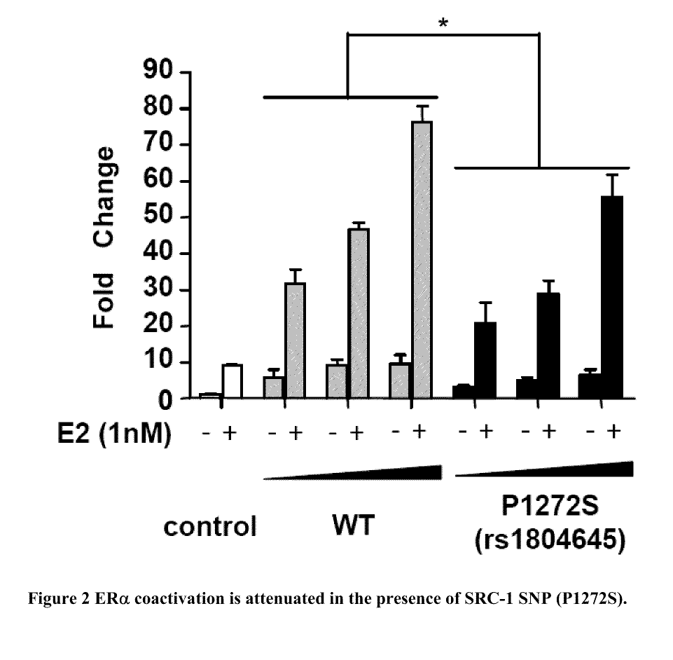

Because the proline to serine substitution resides in the activation domain 2 (AD2) of SRC-1 (FIG. 1), which is known to be critical for its coactivation function¹⁹, we tested whether the P1272S variant demonstrated altered coactivation of ER-alpha. We first performed in vitro studies to determine whether the SRC-1 variant has decreased coactivation potential in a reporter assay using human estrogen responsive cell lines (FIG. 2).

MCF-7 cells were cultured in DMEM culture media (Invitrogen) supplemented with 5% FBS (Hyclone), Penicillin-Streptomycin-Glutamine (Invitrogen). 1.5×105 cells per well were plated in six-well tissue culture plates 48 h prior to transfection using culture medium described above. The medium was then changed to IMEM (Invitrogen) supplemented with 5% charcoal stripped serum (Hyclone) 24 h prior to transfection, and transfections were carried out using Lipofectamine 2000 (Invitrogen) in OPTI-MEM following the manufacturer's protocol. The pSG5-SRC-1 WT expression plasmid was provided by Dr. C. Smith (BCM, Houston, Tex.). The SRC-1 variant P1272S was generated by mutagenesis using the QUICKCHANGEL® Site-directed Mutagenesis Kit (STRATAGENE®) following the manufacturer's protocol.

Cells were transiently transfected with ERE-TK-Luc reporter constructs, and expression constructs for wildtype or P1272S SRC-1. Cells were treated with vehicle or estradiol (10 nM) for 24 h. After harvesting cells using Cell Culture Lysis Reagent 1×(PROMEGA®), Luciferase values were determined using Luminoskan Ascent, Labsystems using Ascent Software 2.6-default. sel. Data was exported and analyzed in excel. Compared to transfection of pSG5 vector alone transfection of Flag-SRC-1 WT or Flag SRC-1 SNP P1272S resulted in a concentration dependent increase of Luciferase activity in the presence of vehicle and especially after Estrogen treatment. A significant loss of co-activation by P1272S SRC-1 compared to WT SRC-1 was observed after Estrogen treatment. The data is presented as fold over control relative to untreated vehicle. Error bars represent standard deviation. *p<0.05 (two-way ANOVA test). Co-transfection of the SRC-1 variant P1272S results in significantly lower ER mediated reporter gene expression as compared to the SRC-1 wildtype in MCF-7 cells. This effect was consistently observed in several additional cell lines including 293, T47D, and Ishikawa cells (data not shown).

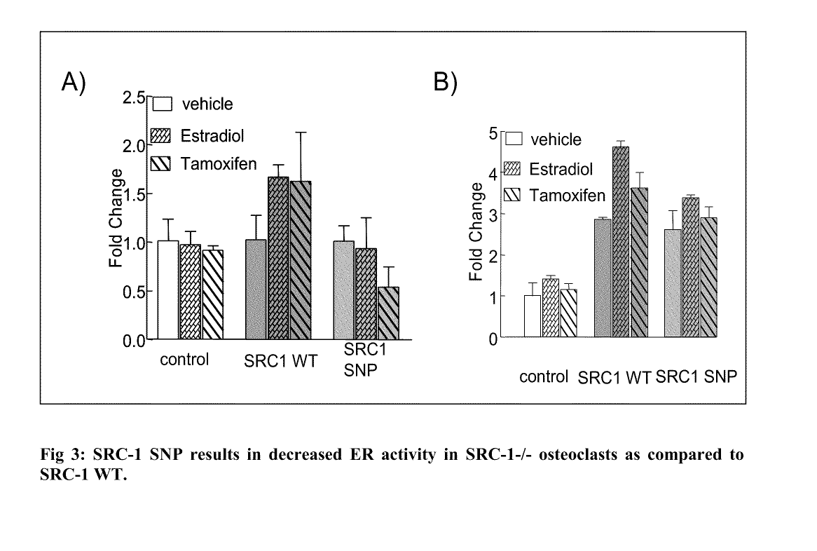

We next compared the co-activation ability of SRC-1 wt and P1272S SRC-1 in osteoclasts derived from SRC-1 −/− mice (FIG. 3). Bone-marrow cells derived from SRC-1−/− mice were plated in 6-well plates containing α-MEM with 10% FBS and 10 ng/ml Macrophage-Colony stimulating factor (M-CSF) (R&D Systems). After incubation for 48 hours, adherent cells were used as osteoclast precursor cells after washing out the nonadherent cells. Cells were cultured in the presence of 10 ng/ml M-CSF and 100 ng/ml RANK ligand (Peprotech) to generate osteoclast-like cells for 7 days. 24 hours before transfection, media was changed to phenol red-free media supplemented with 5% dextran charcoal-stripped serum, 10 ng/ml (M-CSF), and 100 ng/ml RANK ligand. (FIG. 3, A). Mature osteoclasts were obtained from bone marrow of SRC-1−/− mice and cultured in the presence of described concentrations of M-CSF and RANKL. 24 hours before transfection, media was changed to phenol red-free media supplemented with 5% dextran charcoal-stripped serum, M-CSF, and RANKL. Cells were transiently transfected expression constructs for WT or P1272S SRC-1, and treated with vehicle, estradiol (10 nM), or 40H-Tamoxifen (1 μM) for 24 h. Total RNA was isolated using the RNeasy RNA isolation kit including additional DNase treatment (QIAGEN®) as recommended by the supplier. Triplicate RNA samples were independently prepared from each treatment group. 50 ng of total RNA from each sample was reverse transcribed into first-strand cDNA with reverse primers specific for fasl, Pactin, or flag-SRC-1 using Superscript III reverse transcriptase (INVITROGEN®). Real-time RT-PCR was performed using Power SYBR® Green (APPLIED BIOSYSTEMS®) according to the manufacturer's instruction. CT values were determined for fasl, Beta-actin as well as flag-SRC-1 using 7500 Fast realtime PCR System, APPLIED BIOSYSTEMS®. Data was analyzed using 7500 Fast System Software. Furthermore, CT values were exported to excel and further analyzed using the AACT method. Biological triplicate samples were pipetted in duplicate.

Induction of fasl after Estrogen as well as Tamoxifen treatment was detectable in cultured SRC-1−/− osteoclast in the presence of transfected Flag-SRC-1 WT compared to vector alone. However, in the presence of P1272S SRC-1, ER-alpha failed to upregulate fasl after Estrogen as well as Tamoxifen treatment. (FIG. 3, A).

Cells were transiently transfected with ERE-TK-Luc reporter constructs, and expression constructs for wildtype or P1272S SRC-1. Cells were treated with vehicle, estradiol (10 nM), or 40H-Tamoxifen (1 μM) for 24 h. After harvesting cells using Cell Culture Lysis Reagent 1× (PROMEGA®), Luciferase values were determined using Luminoskan Ascent, Labsystems using Ascent Software 2.6-default. sel. Data was exported and analyzed in excel. This time in a SRC-1−/− background, we observed again an increase of Luciferase activity after transfecting Flag-SRC-1 WT or SNP compared to pSG5 alone for all three treatment groups. Transfection of SRC-1 WT resulted in a significant increase of ER activity after treatment with either Estrogen or Tamoxifen, suggesting that Tamoxifen acts as an ER agonist in these cells. In contrast, this Estrogen and Tamoxifen response was impaired in the presence of P1272S SRC-1 (FIG. 3, B).

Estrogen strongly induces osteoclast apoptosis, suggesting that a major mechanism for bone maintenance in premenopausal women is the suppression of osteoclast numbers³. Interestingly, osteopenia with high bone turnover is observed in SRC-1−/− mice, an effect that is similar to that seen in ovariectomized WT mice. Exogenous estrogen is able to reverse the bone loss in ovariectomized WT mice, but not in ovariectomized SRC-1−/− mice, supporting the critical role of SRC-1 in estrogen-dependent bone maintenance. We sought to determine whether rescuing the SRC-1 deficiency in skeletal cell cultures derived from the bone marrow of SRC-1−/− mice with either WT SRC-1 or the P1272S SRC-1 variant would influence the apoptotic response of osteoclasts to estrogen.

Bone marrow cells were isolated from SRC-1−/− mice and plated in six-well tissue culture plates containing a-MEM (INVITROGEN®) with 10% FBS and 10 ng/ml M-CSF (R&D Systems). After incubation for 48 h, cells were cultured for 13 days in the presence of 10 ng/ml M-CSF and 100 ng/ml RANKL (PEPROTECH®) to generate osteoclasts. Following differentiation of bone marrow derived cultures into osteoblasts, pre-osteoclasts, and mature osteoclasts, as previously described^{4,5}, cells were transfected with empty vector (control), SRC-1 WT, or SRC-1 P1272S expression constructs (described above) and then treated with either vehicle or estrogen (10 nM) for 16 hours. Cells were fixed with 4% paraformaldehyde and TUNEL assay (ROCHE™ Applied Sciences) was performed according to the manufacturer's protocol. TRAP staining was performed according to the manufacturer's protocol (SIGMA-ALDRICH®). Apoptosis was detected by determining the ratio of TUNEL-positive cells and DAPI-counterstained cells via fluorescence microscopy. The experiment was performed in triplicate cultures. Student's t-test was performed for SRC-1 WT vehicle versus estradiol (p=0.01) and SRC-1 WT estradiol versus SRC-1 P1272S estradiol (p=0.05). Controls included q-RT-PCR demonstrating equal expression of the exogenous SRC-1 WT and SNP, and TRAP (tartrate-resistant acidic phosphatase) staining showing that the bone marrow cells were successfully differentiated into skeletal cell culture containing approximately 30% TRAP-positive preosteoclasts and osteoclasts (data not shown).

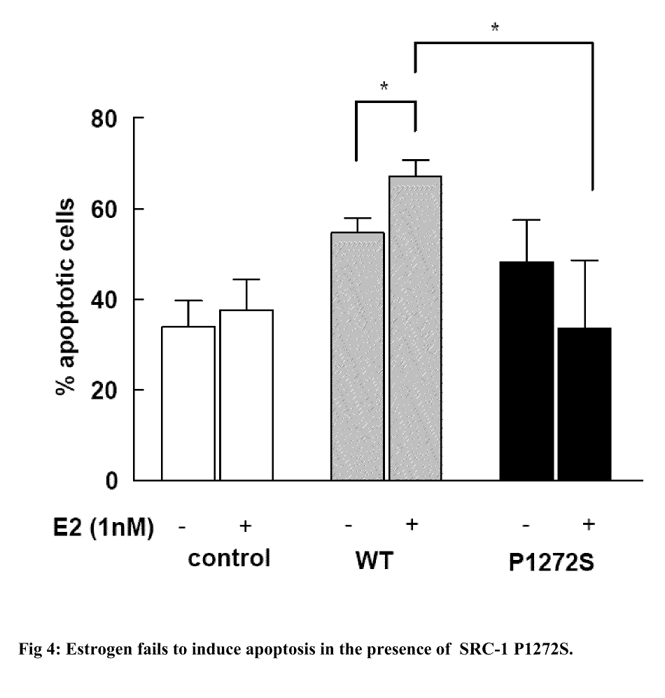

TUNEL assay demonstrated that estrogen increased osteoclast apoptosis in the cells transfected with WT SRC-1, as expected. However, this response was attenuated in the cultures transfected with SRC-1 P1272S as well as in untransfected cultures (FIG. 4). Together, these data strongly suggest that the P1272S attenuates the response of these cells to estrogen, leading to a decrease in osteoclast apoptosis and subsequent increased bone turnover, and therefore serves as a model for the decreased BMD observed in women who carry the SRC-1 P1272S SNP.

To determine whether these in vitro findings could potentially play a role in vivo, we genotyped for the P1272S SRC-1 variant in women enrolled in a prospective cohort clinical trial designed to associate genetic variants with well-curated phenotypic outcomes, including BMD, in response to tamoxifen²⁰.

The registry protocol was approved by the institutional review boards of all participating sites and registered on www.clinicaltrial.gov (NCT00228930). All patients provided written informed consent before entry. Eligible women were recruited into a prospective cohort registry from three breast cancer clinics—the Lombardi Comprehensive Cancer Center at Georgetown University Medical Center, Washington, D.C.; the Breast Oncology Program at the University of Michigan Comprehensive Cancer Center, Ann Arbor, Mich.; and the Indiana University Cancer Center, Indianapolis, Ind. Premenopausal and postmenopausal women (aged 18 years) at high risk for breast cancer, or with newly diagnosed breast cancer who were starting tamoxifen as standard adjuvant therapy were included in this registry. Patients were enrolled after they had completed all primary surgery, radiation, and adjuvant chemotherapy. Since chemotherapy was a strong confounding factor for BMD in this trial (Henry L et. al. unpublished data), we limited our analysis to patients treated exclusively with tamoxifen. Hip and lumbar BMD was measured by DXA scanning before the start of tamoxifen treatment (“baseline”) and after 12 months of treatment (PS207714 and PS207749;www.pharmgkb.org)

All coding exons of SRC-1 were sequenced by Polymorphic DNA (Alameda, Calif.) using germline DNA from 48 Caucasian and 48 African-American individuals (Coriell DNA). For the genotyping studies, germline DNA was extracted from the leukocyte portion of whole blood by use of a QIAAMP® DNA Blood Mini Kit (QIAGEN®, Valencia, Calif.). SRC-1 P1272S (rs1804645), rs2083389, and rs719189 variant alleles were genotyped with a TAQMAN®Allelic Discrimination Assay (APPLIED BIOSYSTEMS®, Foster City, Calif.) according to the manufacturer's instructions. The digested polymerase chain reaction products were then analyzed with an AGILENT® 2100 Bioanalyzer (AGILENT® Technologies, Rockville, Md.).

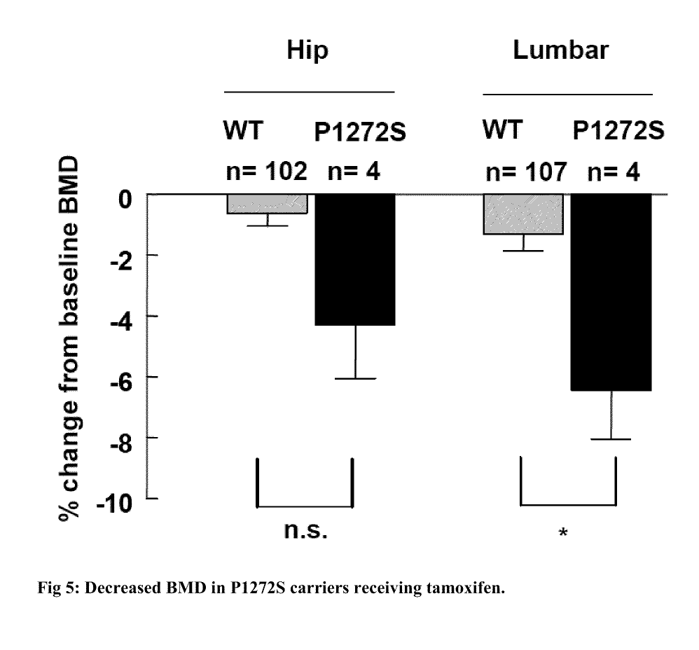

In the majority of the woman, BMD was measured before beginning of treatment (time t=0), and again after 12 months of treatment with tamoxifen. Hip and lumbar BMD was measured by DXA at baseline and after 12 months of tamoxifen treatment. Data for hip and lumbar BMD was available for 106 and 111 patients, respectively. Following 12 months of treatment with tamoxifen, women carrying the P1272S allele showed a significant decrease in BMD compared to women without the variant. Lumbar BMD decreased by 6.4% in P1272S carriers (n=4) versus 1.3% in women without the SNP (n=107) (p=0.04). Similarly, hip BMD decreased by 4.3% for SNP carriers (n=4) versus 0.6% in the WT group (n=102) (p=0.08) (FIG. 5, Error bars represent SEM).

P1272S is located in the activation domain 2 of SRC-1, which contains an ER-alpha binding motif and harbors intrinsic histone acetyl transferase. While not intending to be bound by the following theory, modeling studies predict that the P1272S substitution is probably damaging to the native structure of the protein (http://genetics.bwh.harvard.edu/pph/). Additionally, in silico analysis of the sequence suggests that the variant removes a potential GSK3 (Glycogen synthase kinase 3) target motif at amino acid 1275 (http://scansite.mit.edu/). Intriguingly, phosphorylation by GSK3 has been shown to be involved in modulating the activity and functional lifetime of another member of the SRC family, SRC-322.

The foregoing study examined tamoxifen treated subjects. We next examined the publicly available data from the Framingham Osteoporosis GWAS to pursue possible additional associations between variants in SRC-1 and decreased BMD in the general population. The Framingham Heart Study (FHS) began in 1948 with the recruitment of 5,209 individuals (cohort 1) for analyses relating to common patterns in cardiovascular disease (CVD) development. Subsequently, 2^{nd} and 3^{rd} generation offspring from the original cohort were recruited in two additional cohorts (cohort 2 and 3, respectively). The familial nature of these cohorts and the extensive clinical information associated with them has made them extremely powerful for genetic association studies. Recently, DNA from a subset of individuals in cohort 1 and 2 was used with the Affymetrix 100 k SNP GeneChip to correlate single nucleotide polymorphisms (SNPs) with bone changes, specifically bone mineral density, quantitative ultrasound, and geometric indicies.²⁵

The group conducting the study did extensive genome-wide analysis to identify SNPs associated with bone changes. Two models were used to test for associations: additive generalized estimating equation (GEE) and family-based association tests (FBAT). Each trait was analyzed in a cohort- and sex-specific model. Further, geometric traits were adjusted for age. The 25 most significant SNPs were presented as likely to be associated with decreased BMD. Additionally, they combined traits into groups, analyzed men and women separately, and looked specifically at a list of candidate genes.²⁵

Based on our discovery that the P1272S polymorphism played a significant role in BMD in women, we examined the Framingham Osteoporosis GWAS for confirmation of our findings in the general population. Unfortunately, the P1272S SNP is not represented on the chip used in that study, and insufficient data exists at the present time to determine this polymorphism's Linkage Disequilibrium patterns. The Framingham Osteoporosis GWAS did not identify other SRC-1 associated SNPs as informative for bone density. We none-the-less expanded our search to SNPs associated with SRC-1 that were present in the Framingham Osteoporosis GWAS based on our discovery of the SRC-1 P1272S polymorphism's association with low bone density.

The dataset and analysis from the published FHS association study was made publically available on dbSNP. We downloaded this dataset and extracted femoral neck, lumbar, and trochanter DXA scan association data (analyzed by GEE and FBAT, for men and women analyzed separately and together) for all SNPs within SRC-1 on the chip used in this study (6 total). Surprisingly, two other SRC-1 SNPs, rs2083389 and rs719189, were found to be significantly associated with decreased BMD in women but not men (for both analyses and for most of the DXA measurements) (Table 2). These 2 SNPs are in linkage disequilibrium with each other but not with any of the other SNPs in SRC-1 on the 100 k SNP chip array used in the study. This means that if an individual has the informative allele of one of the 2 SNPs identified, it is highly likely that individual will also have the informative allele of the second SNP. This ‘double-hit’ provides additional evidence a bonefide association has been made.

TABLE 2
SNPs in SRC-1 associate with low baseline BMD in FHS 100 k dataset.

| | | | Women | | Men | | All | |
|---|---|---|---|---|---|---|---|---|
| | | | # of | p- | # of | p- | # of | p- |
| SNP | MAF | Measurement | families | value | families | value | families | value |
| rs2083389 | 0.23 | Femoral Neck | 110 | 0.002 | 90 | 0.330 | 138 | 0.060 |
| | | Lumbar Spine | 117 | 0.010 | 94 | 0.900 | 137 | 0.030 |
| | | Trochanter | 118 | 0.005 | 95 | 0.320 | 138 | 0.090 |
| rs719189 | 0.18 | Femoral Neck | 97 | 0.006 | 75 | 0.610 | 124 | 0.050 |
| | | Lumbar Spine | 103 | 0.070 | 80 | 0.950 | 123 | 0.110 |
| | | Trochanter | 104 | 0.008 | 81 | 0.550 | 124 | 0.050 |

Data shown in Table 2 represent Family Based Association Tests (FBAT)^{23,24} in women, men, and all subjects. (MAF=minor allele frequency). Associations between SRC-1 genotypes (rs2083389, and rs719189) and baseline lumbar and hip BMD were examined in each menopausal group. The comparisons were performed using linear regression within each menopausal status. Associations between SRC-1 genotypes and the percent changes in lumbar and hip BMD from baseline to month 12 were assessed by using a general linear model (GLM) and adjusted for center. GLM was performed using the SAS procedure (PROC GLM, SAS v9.1.3). For post-hoc comparisons, we compared the adjusted means between all pairs of three genotypes while controlling for overall alpha. For all analyses, a p-value of less than 0.05 was considered statistically significant.

The association between the SNPs and BMD was highly significant in females but was not present in males, closely mirroring the results seen in SRC-1−/− mice, in which only females, but not males, are resistant to the skeletal response induced by estradiol after gonadectomy²¹. Further, within the HapMap populations, these two intronic SNPs are in LD with each other thus providing strong evidence of a bona fide association. Importantly, these SNPs have minor allele frequencies (MAF) between 18-23% and thus represent a large portion of the population.

Despite the above results, we were concerned that the association identified above was not identified in the original study. One possible explanation is that genome-wide studies contain a vast amount of data which make it extremely difficult to discern real effects from statistical noise. Because of the vast amount of data and statistical variance inherent therein, the investigators examining the FHS dataset for BMD associations used stringent p-value criteria. They reported, as most likely significant, the top 25 SNPs (by p-value) for all SNPs tested without regard to gender. The SRC-1 SNPs' (rs2083389 and rs719189) correlation to BMD were well below the cut-off p-value of 1×10⁻³, and thus were not listed in their analysis.

One advantage of our candidate gene driven focus on SRC-1 SNPs was the knowledge that gender stratification of the data set would potentially provide a stronger statistical association with BMD. When we examined female data, the SRC-1 SNPs we found (rs2083389 and rs719189) were ranked 7/9198 and 29/9198 for femoral neck BMD using the same FBAT analysis applied the full dataset in the original FHS study. This suggested that the associations between these SRC1 SNPs and BMD loss are very strong in females. Again, these associations were still well below the original FHS study's cut-off p-value (1.00×10⁻³ versus 1.78×10⁻³ and 6.18×10⁻³), applied to screen out false positives from the genome wide analysis.

It is clear that the original analysis of this dataset did not identify rs2083389 and rs719189 to be associated with a bone phenotype. Because the FHS study was making comparisons across the entire genome, very strict p-value limits had to be implemented to protect against false-positives. Our approach, on the other hand, was to focus the dataset to a candidate gene (SRC-1/NCoA1) by using our pre-existing evidence that SRC-1 is important for bone health. This candidate gene approach therefore yielded data using the FHS dataset that could not otherwise be uncovered from the inherent statistical noise in the previous genome wide study.

By using a candidate gene approach, we were not subjected to the same statistical constraints typical of a genome-wide study and thus were able to identify rs2083389 and rs719189 as additional SRC-1 SNPs which are useful for assessing the risk of BMD problems in women. Importantly, rs2083389 and rs719189 are present at very high frequencies in the HapMap populations ranging from 9-24%. Thus, genotyping for these SNPs could potentially identify a large portion of women at risk for osteoporosis.

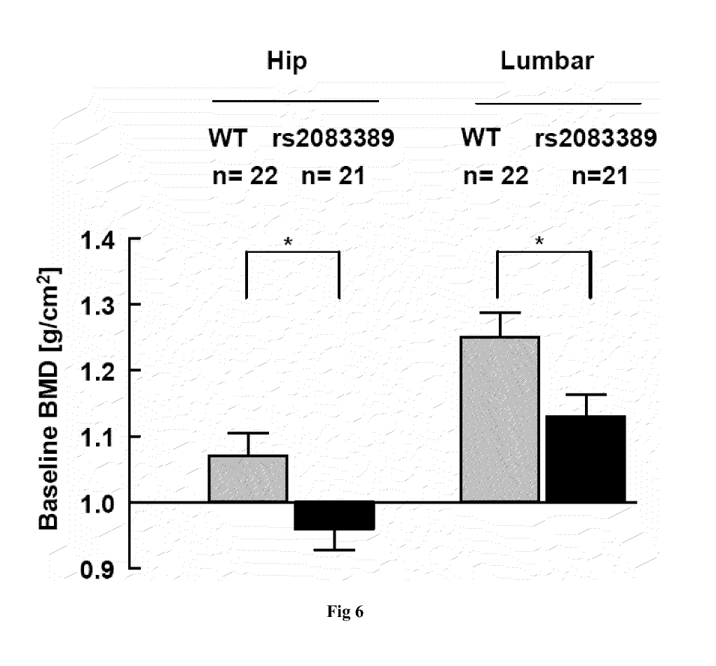

Because the rs2083389 and rs719189 identification was unexpected from a previously analyzed dataset, we attempted to validate these findings by determining the rs2083389 and rs719189 genotypes within our COBRA trial patient cohort (described above). Consistent with the association we observed in the FHS dataset, both SNPs were associated with lower baseline BMD (FIG. 6), thus validating the association in an independent population. Interestingly, this association was only seen in premenopausal but not in postmenopausal women in the COBRA cohort suggesting that P1272S (rs1804645) and rs2083389/rs719189 represent two distinct functional alterations within the SRC-1 locus, which influence bone health maintenance through different mechanisms.

Consistent with the distinction in populations affected, our data show that P1272S (rs1804645) is not in LD with the rs2083389/rs719189 SNPs identified using the Framingham Osteoporosis GWAS dataset. Thus, these three SRC-1 variants (rs1804645, rs2083389, and rs719189) most likely represent two distinct functional variants. Specifically, rs 1804645 represents one such functional variant and likely is the causative protein polymorphism (P1272S). At this point it is not yet determined whether rs2083389 and/or rs719189 are themselves functional variants or if they are in LD with a yet uncharacterized variant.

This data provides additional evidence that SRC-1 plays a critical role in BMD. Thus, detection of SRC-1 variants (SNPs) may be used to predict increased risk for bone mineral loss, and thus identify women who may require close monitoring and subsequent early intervention with alternative bone-preserving agents if necessary.

The following references and those cited elsewhere within the Specification are hereby incorporated by reference in their entireties and, in particular, for any disclosure for which the references are specifically relied upon:
1. Cummings, S. R. & Melton, L. J. Epidemiology and outcomes of osteoporotic fractures. The Lancet 359, 1761-1767 (2002).
2. Osteoporosis prevention, diagnosis, and therapy. NIH Consens Statement 17, 1-45 (2000).
3. Rodan, G. A. & Martin, T. J. Therapeutic Approaches to Bone Diseases. Science 289, 1508-1514 (2000).
4. Krum, S. et al. Estrogen protects bone by inducing Fas ligand in osteoblasts to regulate osteoclast survival. Embo Journal 27, 535-545 (2008).
5. Nakamura, T. et al. Estrogen Prevents Bone Loss via Estrogen Receptor [alpha] and Induction of Fas Ligand in Osteoclasts. Cell 130, 811 (2007).
6. Peacock, M., Turner, C. H., Econs, M. J. & Foroud, T. Genetics of Osteoporosis. Endocr Rev 23, 303-326 (2002).
7. Richards, J. B. et al. Bone mineral density, osteoporosis, and osteoporotic fractures: a genome-wide association study. The Lancet 9 (2008).
8. Styrkarsdottir, U. et al. Multiple Genetic Loci for Bone Mineral Density and Fractures. N Engl J Med, NEJMoa0801197 (2008).
9. Kiel, D. et al. Genome-wide association with bone mass and geometry in the Framingham Heart Study. BioMed Central Medical Genetics 8 (2007).
10. Toannidis, J. P. A. et al. Differential Genetic Effects of ESR1 Gene Polymorphisms on Osteoporosis Outcomes. JAMA 292, 2105-2114 (2004).
11. Xu, J. & O'Malley, B. W. Molecular mechanisms and cellular biology of the steroid receptor coactivator (SRC) family in steroid receptor function. Rev Endocr Metab Disord 3, 185-92 (2002).
12. Onate, S. A., Tsai, Sophia Y., Tsai, Ming-Jer, O'Malley, Bert W. Sequence and Characterization of a Coactivator for the Steroid Hormone Receptor Superfamily. Science 270, 1354-1357 (1995).
13. Xu, J. & Li, Q. Review of the in Vivo Functions of the p160 Steroid Receptor Coactivator Family. Mol Endocrinol 17, 1681-1692 (2003).
14. Smith, C. L., Nawaz, Z. & O'Malley, B. W. Coactivator and Corepressor Regulation of the Agonist/Antagonist Activity of the Mixed Antiestrogen, 4-Hydroxytamoxifen. Mol Endocrinol 11, 657-666 (1997).
15. Shang, Y. & Brown, M. Molecular Determinants for the Tissue Specificity of SERMs. Science 295, 2465-2468 (2002).
16. Jordan, V., Phelps, E. & Lindgren, J. Effects of anti-estrogens on bone in castrated and intact female rats. Breast Cancer Res Treat 10, 31-35 (1987).
17. Moedder, U. I. L. et al. Effects of Loss of Steroid Receptor Coactivator-1 on the Skeletal Response to Estrogen in Mice. Endocrinology 145, 913-921 (2004).
18. Yamada, T. et al. SRC-1 Is Necessary for Skeletal Responses to Sex Hormones in Both Males and Females. Journal of Bone and Mineral Research 19, 1452-1461 (2004).
19. Kalkhoven, E., Valentine, J. E., Heery, D. M., Parker, M. G. Isoforms of steroid receptor co-activator 1 differ in their ability to potentiate transcription by the oestrogen receptor. EMBO Journal 17, 232-243 (1998).
20. Jin, Y. et al. CYP2D6 genotype, antidepressant use, and tamoxifen metabolism during adjuvant breast cancer treatment. J Natl Cancer Inst 97, 30-9 (2005).
21. Mödder, U. I. et al. The skeletal response to estrogen is impaired in female but not in male steroid receptor coactivator (SRC)-1 knock out mice. Bone 42, 414-421 (2008).
22. Wu, R. C., Feng, Q., Lonard, D. M. & O'Malley, B. W. SRC-3 coactivator functional lifetime is regulated by a phospho-dependent ubiquitin time clock. Cell 129, 1125-40 (2007).
23. Cupples, L. A. et al. The Framingham Heart Study 100K SNP genome-wide association study resource: overview of 17 phenotype working group reports. BMC Medical Genetics 8, S1 (2007).
24. Rabinowitz, D. & Laird, N. A unified approach to adjusting association tests for population admixture with arbitrary pedigree structure and arbitrary missing marker information. Hum Hered 50, 211-223 (2000).
25. Kiel, D. P., et al. Genome-wide association with bone mass and geometry in the Framingham Heart Study. BMC medical genetics 8 Suppl 1, S14 (2007).

1. A method of detecting a nucleic acid polymorphism in SRC-1 comprising the step of detecting an allele of
a) a nucleic acid polymorphism in linkage disequilibrium (LD) with the SRC-1 nucleic acid polymorphism, or
b) the nucleic acid polymorphism in SRC-1.

2. The method of claim 1 comprising the step of analyzing a nucleic acid polymorphism in linkage disequilibrium (LD) with the SRC-1 nucleic acid polymorphism, wherein the polymorphism in linkage disequilibrium (LD) with the SRC-1 nucleic acid polymorphism belongs to a haplotype containing the nucleic acid polymorphism in SRC-1.

3. The methods of claims 1, wherein the polymorphism linkage disequilibrium (LD) with the SRC-1 nucleic acid polymorphism is a SNP.

4. The method of claim 1 comprising the step of analyzing the nucleic acid polymorphism in SRC-1, wherein the nucleic acid polymorphism in SRC-I is a SNP.

5. The method of claim 1, wherein the allele of the nucleic acid polymorphism in SRC-1 encodes a Serine at amino acid 1272 of the protein encoded by SRC-1, or at an equivalent amino acid position in a homologue of SRC-1.

6. The method of claim 5, wherein the nucleic acid polymorphism in SRC-1 is an SNP rs1804645.

7. The method of claims 1, wherein the nucleic acid polymorphism in SRC-1 is an SNP rs2083389 and/or an SNP rs719189.

8. The method of claims 1 wherein the nucleic acid comprising SRC-1 is derived from a blood sample or other bodily tissue sample.

9. The method of claims 1 comprising the step of amplifying a genomic DNA comprising the SRC-1 nucleic acid polymorphism, the amplified genomic DNA optionally further including the nucleic acid polymorphism in linkage disequilibrium (LD) with the SRC-1 nucleic acid polymorphism.

10. The method of claim 9 wherein the step of amplifying the genomic DNA comprises a polymerase chain reaction.

11. A method of determining the risk of osteopenia or osteoporosis in a subject comprising the steps of:
a) detecting in the subject, a nucleic acid polymorphism in SRC-1 according to the method of any of claims 1-10, and
b) correlating the detected allele with a risk of the subject developing osteopenia or osteoporosis.

12. The method of claim 11, wherein the subject is human.

13. The method of claim 11, wherein the subject is female.

14. The method of claim 11, wherein the subject has been or will be exposed to an antiestrogen.

15. The method of claim 12, wherein the human subject a) has breast or prostate cancer and b) has been or will be exposed to an antiestrogen.

16. The method of claim 14, wherein the antiestrogen is tamoxifen, raloxifene, goserelin acetate, leuprolide acetate, megestrol, toremifene, fulvestrant; a nonsteroidal or a steroidal aromatase inhibitor including, for example, exemestane, anastrozole and letrozole.

17. A method of screening an SRC-1 protein variant for modulation of a coactivation potential of the variant comprising the step of measuring a transcription of a nucleic acid, or a protein expression from the transcription of the nucleic acid, in the presence of the SRC-1 protein variant.

18. The method of claim 17, wherein the nucleic acid and SRC-1 protein variant are in a cell, cell lysate, or cell lysate fraction.

19. The method of claim 18 wherein the nucleic acid and SRC-1 protein variant are in an estrogen responsive cell.

20. The method of claim 19, further comprising the step of exposing the estrogen responsive cell to estradiol, tamoxifen, raloxifene, goserelin acetate, leuprolide acetate, megestrol, toremifene, fulvestrant; a nonsteroidal or a steroidal aromatase inhibitor including, for example, exemestane, anastrozole and letrozole.

21. The methods of claim 17, wherein the nucleic acid comprises a reporter gene and the nucleic acid is capable of being transactivated by an estrogen receptor.

22. The method of claim 19 wherein the cell is an MCF-7 cell, an SRC-1−/− osteoclast cell or an SRC-1−/− osteoclast-like cell.

23. The method of claim 21, wherein the reporter gene encodes a luciferase.

24. The method of claim 17 further comprising the steps of:
a) measuring a transcription of a nucleic acid, or a protein expression from the transcription of the nucleic acid, in the presence of a SRC-1 protein having a Proline at amino acid 1272, and
b) comparing the measurements for the SRC-1 protein having a Proline at amino acid 1272 and the measurements for the SRC-1 protein variant to determine the modulation of the coactivation potential of the SRC-1 protein variant.

25. The method of claim 17, further comprising the steps of:
a) screening the SRC-1 protein variant for modulation of the coactivation potential in the presence of an additional compound, and
b) determining whether the additional compound modulates the coactivation potential of the SRC-1 protein variant.

26. A method of screening an SRC-1 protein variant for a modulation of a coactivation potential of the variant comprising the step of measuring an expression of a Fas ligand on a cell, the cell comprising an SRC-1 protein variant.

27. The method of claim 26, further comprising the steps of:
a) measuring an expression of a Fas ligand on a second cell, the second cell comprising an SRC-1 protein having a Proline at amino acid 1272, and
b) comparing the measurements for the SRC-1 protein having a Proline at amino acid 1272 and the SRC-1 protein variant to determine the modulation of the coactivation potential of the SRC-1 protein variant.

28. The method of claim 26, further comprising the step of exposing the cell to estradiol, tamoxifen, raloxifene, goserelin acetate, leuprolide acetate, megestrol, toremifene, fulvestrant; a nonsteroidal or a steroidal aromatase inhibitor including, for example, exemestane, anastrozole and letrozole.

29. The method of claim 26 wherein the cell is an SRC-1−/− osteoclast cell or an SRC-1−/− osteoclast-like cell.

30. The method of claim 26, further comprising the steps of:
a) screening the SRC-1 protein variant for modulation of the coactivation potential in the presence of an additional compound, and
b) determining whether the additional compound modulates the coactivation potential of the SRC-1 protein variant.
